SEQUENCE LISTING (7 sequences)

SEQ ID NO: 1 - DNA, 20 bp, Artificial Sequence
  NF-kB decoy
ccttgaagggatttccctcc

SEQ ID NO: 2 - DNA, 28 bp, Artificial Sequence
  STAT-1 decoy
gatctagggatttccgggaaatgaagct

SEQ ID NO: 3 - DNA, 16 bp, Artificial Sequence
  GATA-3 decoy
agcttgagatagagct

SEQ ID NO: 4 - DNA, 28 bp, Artificial Sequence
  STAT-6 decoy
gatcaagaccttttcccaagaaatctat

SEQ ID NO: 5 - DNA, 19 bp, Artificial Sequence
  AP-1 decoy
agcttgtgagtcagaagct

SEQ ID NO: 6 - DNA, 20 bp, Artificial Sequence
  Ets decoy
aattcaccggaagtattcga

SEQ ID NO: 7 - DNA, 14 bp, Artificial Sequence
  E2F decoy
ctagatttcccgcg

(19) United States
(12) Patent Application Publication
(10) Pub. No.: US 2011/0009470 A1
(43) Pub. Date: Jan. 13, 2011

(54) Nucleic acid external skin formulation

(75) Inventor: Masaya Tanaka, Hyogo (JP)

Correspondence Address: FLYNN THIEL BOUTELL & TANIS, P.C., 2026 RAMBLING ROAD, KALAMAZOO, MI, 49008-1631, US

(73) Assignee: NEOCHEMIR INC.

(21) Appl. No.: 12/800,575

(22) Filed: May 18, 2010

Related U.S. Application Data
(62) Division of application No. 11/732,240, filed on Apr. 3, 2007, now abandoned.

(51) Int. Cl. A61K 31/713; A61K 31/7088; A61P 17/06; A61P 29/00

(52) U.S. Cl. 514/44 A; 514/44 R

(57) ABSTRACT

The present invention provides a nucleic acid external skin formulation having a high skin permeability and which is prepared from a polymeric nucleic acid having a high molecular weight and sodium alginate. The nucleic acid external skin formulation is highly skin permeable and delivers its active ingredient nucleic acid to the affected area efficiently. The nucleic acid used may be smaller in amount, and thus, the formulation is also superior in safety and cost. The working mechanism thereof is different from that of the low-molecular weight compounds currently used as an active ingredient for skin anti-inflammatory agents, and thus, the formulation may be applicable to cases where such low-molecular weight medicines are less effective.

This is a division of Ser. No. 11/732 240, filed Apr. 3, 2007.

### BACKGROUND OF THE INVENTION

1. Field of the Invention

The present invention relates to a pharmaceutical formulation containing a nucleic acid, and in particular, to an external skin formulation containing an oligonucleotide.

2. Description of the Related Art

It is effective to control the permeability of the active ingredients (primary ingredients) properly for an improvement of the therapeutic effect of a coating formulation such as ointment or lotion. For example, it is necessary to make the primary ingredient penetrate into the epithelium and dermis of the skin structure in many skin diseases, and the so-called skin barrier in the keratinous-epithelial structure inhibits penetration as the barrier. Physical chemical properties needed for a primary ingredient to easily penetrate the skin barrier are a high oil solubility and low molecular weight (molecular weight up to about 800). For example, steroids and vitamin D, which are known to pass through the skin barrier efficiently, are relatively lower in molecular weight and highly oil-soluble. On the contrary, highly water-soluble and high-molecular weight molecules are known to be less penetrative through the skin barrier. Even after formulation studies, there was almost no formulation sufficiently permeable.

Along with recent progress in biotechnology, there are increasingly more studies aimed at developing new medicines for treating a disease by controlling gene expression. One of these medicines are nucleic acid drugs, for example, containing an oligonucleotide, but most of the oligonucleotides are polymers having a molecular weight of 5,000 or more and are extremely water-soluble. As described above, there was no formulation having superior skin permeability, for example, in developing a coating formulation for skin diseases such as atopic dermatitis containing such a highly water-soluble and high-molecular weight nucleic acid medicine as the primary ingredient, and thus, there exists a need for the development thereof.

Skin diseases with the affected region more exposed and apparent are diseases causing not only physical damage but also mental damage. For example, atopic dermatitis, whose morbidity rate is increasing, often leads affected areas widely spread over the body and is hard to cure, and thus, the patients suffer for an extended period of time. It is an allergic disease affecting a wide range of patients from children to adults. The skin has a function as a barrier of protecting the body from external substances and stimuli, but in patients with atopic dermatitis, deterioration in the barrier function of the skin leads to easier penetration of environmental antigens, such as ticks, and consequently to an allergic reaction. However, steroid ointments commonly used for treatment of the atopic dermatitis suppress the allergic reaction but also deteriorate the skin barrier function, and thus, are not suited for long-term use.

An external skin formulation shows its therapeutic effect after penetration through the skin, and thus, its primary ingredient should penetrate into the skin through the barrier. However, when the active ingredient of the external skin formulation is, for example, a macromolecule such as nucleic acid, it was further more difficult to make the ingredient penetrate into the skin. For that reason, it was difficult to develop an external skin formulation containing a macromolecule such as nucleic acid as the active ingredient.

In such a background, there exists a need for development of a medicine that penetrates into the skin despite the skin barrier function described above and shows its therapeutic action.

After intensive studies to solve the problems above, the inventors have found unexpectedly that a viscous polysaccharide is superior as the penetration-accelerator and also that alginic acid or the pharmacological salt thereof, which is used as a moisturizer in cosmetics, is most effective, and achieved the present invention.

Alginic acid is a polysaccharide derived from seaweed, and has been used as a thickener, coating agent, or moisturizer in foods and cosmetics, but the action thereof as a penetration-accelerator had not been known at all.

Japanese Patent Publication No. 2001-521887 discloses a formulation containing a non-steroidal anti-inflammatory agent (NSAID) and a polysaccharide gum such as alginate. However, although the formulation is a mixture of a low-molecular weight compound and an alginate (an alginate salt), the skin permeability of a high-molecular-weight compound such as nucleic acid was not discussed in the patent application.

Patent Publication No. WO 02/066,077 discloses a slow-release formulation for lung diseases comprising a combination of alginic acid and an oligonucleotide. The formulation comprising the combination of alginic acid and a nucleic acid is a medicine for lung diseases, and the skin permeability was not described therein.

Recently, gene drugs, or nucleic acid drugs, are attracting greater attention, and there are many gene therapies studied. However, there is no established nucleic acid drug for treatment of skin diseases, and such a drug, if developed, may be applicable to diseases to which no conventional medicine was effective.

### SUMMARY OF THE INVENTION

An object of the present invention is to provide a skin external formulation containing a nucleic acid as its active ingredient. In particular, the external formulation should preserve the skin barrier function and yet allow effective penetration of the nucleic acid into the skin.

The inventors have found that, in a mixed formulation containing a nucleic acid-based medicine such as NF-κB decoy oligonucleotide and sodium alginate, the oligonucleotide shows high skin permeability. Thus, the present invention provides a highly skin-permeable external skin formulation containing a nucleic acid as the active ingredient.

In general, it provides:
(1) A pharmaceutical composition for delivery of a nucleic acid-based medicine into the cells in dermal or epidermal structure, comprising a nucleic acid-based medicine and a viscous polysaccharide;
(2) The composition according to (1), wherein the nucleic acid-based medicine is a gene or an analogue thereof;
(3) The composition according to (1) or (2), wherein the gene or the analogue thereof is a polynucleotide or oligonucleotide;
(4) The composition according to (1) to (3), wherein the oligonucleotide is a decoy (decoy molecule), antisense, ribozyme, aptamer or siRNA;
(5) The composition according to (1) to (4), wherein the decoy has a transcription factor-inhibiting action;
(6) The composition according to (1) to (5), wherein the decoy is a NF-κB, STAT-1, GATA-3, STAT-6, AP-1, Ets, or E2F decoy oligonucleotide;
(7) The composition according to (1) to (6), wherein the decoy is the NF-κB decoy oligonucleotide represented by SEQ ID No. 1;
(8) The composition according to (1) to (7), wherein the composition contains the nucleic acid-based medicine at 0.01 to 5 mass %;
(9) The composition according to (1) to (8), wherein the composition contains the nucleic acid-based medicine at 0.1 to 1 mass %;
(10) The composition according to (1) to (9), wherein the viscous polysaccharide is alginic acid or the pharmacologically allowable salt thereof;
(11) The composition according to (1) to (10), wherein the viscous polysaccharide is sodium alginate;
(12) The composition according to (1) to (11), wherein the composition contains the viscous polysaccharide at 0.01 to 10 mass %;
(13) The composition according to (1) to (12), wherein the composition contains the viscous polysaccharide at 0.1 to 5 mass %;
(14) The composition according to (1) to (13), wherein the composition further contains a phosphate salt;
(15) The composition according to (1) to (14), wherein the phosphate salt is sodium dihydrogen phosphate;
(16) The composition according to (1) to (15), wherein the composition contains the phosphate salt at 0.01 to 10 mass %;
(17) the composition according to (1) to (16), wherein the composition contains the phosphate salt at 0.1 to 5 mass %;
(18) A therapeutic agent for prophylaxis, amelioration and treatment of a skin disease, comprising the composition according to (1) to (17); and
(19) The therapeutic agent for prophylaxis, amelioration and treatment of a skin disease according to (18), wherein the skin disease is atopic dermatitis, contact dermatitis, photosensitive dermatitis, appendicular chronic dermatitis, seborrheic dermatitis, nummular dermatitis, systemic exfoliative dermatitis, stasis dermatitis, local abrasion dermatitis, medicamentosus dermatitis, or psoriasis.

The nucleic acid external skin formulation according to the present invention, which is highly skin permeable, delivers its active ingredient nucleic acid to the affected region effectively. Because of its high skin permeability, the nucleic acid may be administered at low dosage, and thus, the formulation becomes more safe and economical. The formulation is different from low-molecular-weight medicines, such as anti-inflammatory agents currently used in its working mechanism, and thus, may be effective for the diseases to which the low-molecular-weight medicine show no advantageous effect.

### BRIEF DESCRIPTION OF THE DRAWINGS

FIG. 1 is a chart showing the results of a skin permeability test of NF-κB decoy oligonucleotide by using rat normal skin, in which formulation A and aqueous formulation A are compared with each other;

FIG. 2 is a chart showing the results of a skin permeability test of NF-κB decoy oligonucleotide by using rat damaged skin, in which formulation A and aqueous formulation A are compared with each other;

FIG. 3 is a view illustrating the coating sites in the cumulative rabbit skin irritation test according to Draize method, and is a view of the back of a rabbit, as seen from above;

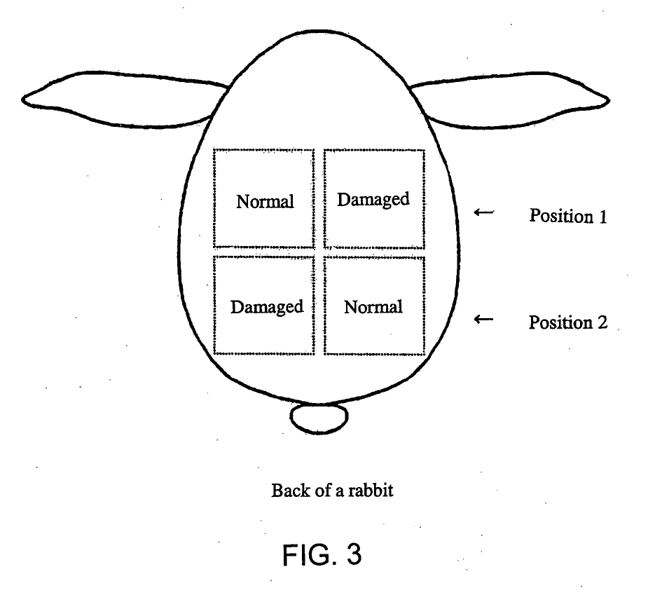

FIG. 4 is a chart showing the average of the erythema and edema score with time at a site of a rabbit where the NF-κB decoy oligonucleotide was administered, in which normal and damaged skins are compared with each other after only the base substance of aqueous formulation A (without nucleic acid) is administered thereon, wherein the error bar represents the standard deviation; and

FIG. 5 is a chart showing the average of the erythema and edema score with time at a site of a rabbit where the NF-κB decoy oligonucleotide was administered, in which normal and damaged skins are compared with each other after the aqueous formulation A is administered, wherein the error bar in the figure represents the standard deviation.

### DESCRIPTION OF THE PREFERRED EMBODIMENTS

The present invention provides a pharmaceutical composition containing a nucleic acid-based medicine and a penetration-accelerator for delivery of a nucleic acid-based medicine into the cells in dermis or epidermis.

The nucleic acid-based medicines for use in the present invention normally include decoys, antisenses, ribozymes, aptamers, siRNAs, and the like, but preferably decoys. The decoy is a double-stranded oligonucleotide that competes with a nucleic acid binding sequence when a transcriptional regulatory factor binds to the binding sequence. The terms, decoy, decoy oligonucleotide, and decoy ODN, are used synonymously. One or more nucleic acid-based medicines may be used in combination, and are favorably used in an amount of 0.01 to 5 mass % (hereinafter, indicated simply by “%”), in particular 0.1 to 1%, in the external skin formulation according to the present invention.

Examples of the nucleic acid-based medicines according to the present invention include decoys such as NF-κB, STAT-1, GATA-3, STAT-6, AP-1, Ets, and E2F; and NF-κB is suitable. Preferable examples of the decoys include 5′-ccttgaagggatttccctcc-3′ (SEQ ID No. 1) (NF-κB decoy), 5′-gatctagggatttccgggaaatgaagct-3′ (SEQ ID No. 2) (STAT-1 decoy), 5′-agcttgagatagagct-3′ (SEQ ID No. 3) (GATA-3 decoy), 5′-gatcaagaccttttcccaagaaatctat-3′ (SEQ ID No. 4) (STAT-6 decoy), 5′-agcttgtgagtcagaagct-3′ (SEQ ID No. 5) (AP-1 decoy), 5′-aattcaccggaagtattcga-3′ (SEQ ID No. 6) (Ets decoy), 5′-ctagatttcccgcg-3′ (SEQ ID No. 7) (E2F decoy), the oligonucleotides containing the complementary sequence, the mutants thereof, and the compounds containing any of them in the molecule. The oligonucleotide may be DNA or RNA or alternatively, may contain a modified nucleic acid and/or a pseudonucleic acid in the oligonucleotide. The nucleic acid-based medicine is, for example, a double-stranded oligonucleotide or the mutant thereof containing one or more of its nucleic acid sequences.

For example, a viscous polysaccharide or a phosphate salt is used as the penetration-accelerator according to the invention; preferably, a viscous polysaccharide and a phosphate salt are used alone or in combination; and more preferably, a mixture of a viscous polysaccharide and a phosphate salt is used.

Examples of the viscous polysaccharides normally include alginic acid, laminalan, carrageenan, furusefuran, porphyran, gurateropiran, gurusan, fucoidan, hyaluronic acid, and the like, and alginic acid, and carrageenan are preferable; and alginic acid is more preferable. The alginic acid includes alginic acid and the pharmacologically allowable salts thereof, and is normally, preferably sodium alginate. The alginic acid may be a commercially available product such as “Kimica Algin” (manufactured by Kimica Corporation).

These viscous polysaccharides may be used alone or as a mixture of two or more, and are preferably added to the external skin formulation according to the present invention in an amount of 0.01 to 10 mass % (hereinafter, indicated simply by “%”), in particular 0.1 to 5%.

Examples of the phosphate salts normally include sodium dihydrogen phosphate, calcium hydrogen phosphate, tripotassium phosphate, tricalcium phosphate, diammonium hydrogen phosphate, dipotassium hydrogen phosphate, disodium hydrogen phosphate, trisodium phosphate, calcium dihydrogen phosphate, and the like, and sodium dihydrogen phosphate is used preferably. These phosphate salts may be used alone or as a mixture of two or more, and are preferably contained in the external skin formulation according to the present invention in an amount of 0.01 to 10 wt %, in particular 0.1 to 5%.

In addition to the essential components above, ingredients used in medicines and cosmetics such as water, oils, waxes, silicones, surfactants, alcohols, polyvalent alcohols, water-soluble polymer thickeners, pH-adjusters, flavoring agents, antioxidants, chelating agents, colorants, pigments, antiseptics, other pharmaceutical components, and ultraviolet absorbents, and also other inorganic and organic ingredients may be added as needed to the external skin formulation according to the present invention in a range that does not impair the advantageous effects of the present invention qualitatively or quantitatively.

Examples of the diseases to be treated with the external skin formulation according to the present invention include skin diseases such as atopic dermatitis, contact dermatitis, photosensitive dermatitis, appendicular chronic dermatitis, seborrheic dermatitis, nummular dermatitis, systemic exfoliative dermatitis, stasis dermatitis, local abrasion dermatitis, medicamentosus dermatitis or psoriasis, and the like.

The external skin formulation according to the present invention is prepared with the essential components and as needed with any other ingredients according to a common production method, in various formulation forms such as a liquid, lotion, oil, cream, and ointment.

The dosage may vary according to the age, body weight, symptom of the patient, treatment efficiency, administration method, treatment period, and others, but the external formulation is normally applied in an amount sufficient for covering the affected area, one to three time(s) a day.

Hereinafter, the present invention will be described specifically with Examples, but it should be understood that the present invention is not restricted thereby.

### Examples

1. Skin Permeability Test 1-1.

For evaluation of the skin permeability of a NF-κB decoy oligonucleotide by single percutaneous administration in a rat, the radioactivity concentration in the blood or skin was determined by using a ³⁵S-labelled ³⁵S-NF-κB decoy oligonucleotide (hereinafter, referred to as ³⁵S-NF-κB decoy).

The chemical structure and the labeling site of ³⁵S-NF-κB decoy are shown below:

³⁵S-5′-CCTTGAAGGGATTTCCCTCC-3′
³⁵S-3′-GGAACTTCCCTAAAGGGAGG-5′

The ³⁵S-NF-κB decoy was prepared according to the method of Agrawal et al., (Proc. Natl. Acad. Sci. USA, 88, 7595-7599, 1991). The H-phosphonated oligonucleotide was made, and H in the H-Phosphonate was oxidized to ³⁵S with ³⁵S₈ (elemental sulfur) before deprotection of the H-phosphonated oligonucleotide; and the product was deprotected in a common deprotection reaction, to give an intended ³⁵S-labelled oligonucleotide.

(1) Preparation of Administration Formulation

A certain amount of ³⁵S-NF-κB decoy stock solution and an unlabelled NF-κB decoy were placed on an agate mortar or in a plastic container, and a large excess amount of ethanol was added thereto, to precipitate the NF-κB decoy. The supernatant was then removed, and the solvent was removed completely under a nitrogen gas flow, to give crude ³⁵S-NF-κB decoy.

Formulation A (containing only vaseline)

| | | | |
|---|---|---|---|
| ³⁵S-NF-kB decoy | 0.5% | 2.0 | mg |
| Stearyl alcohol | 5.0% | 20.0 | mg |
| White vaseline | 94.5% | 378.0 | mg |
| Total | 100.0% | 400.0 | mg |

Certain amounts of stearyl alcohol and white vaseline were melt-blended in a water bath at 70 to 80° C. and then cooled to room temperature, to give a mixed base substance. A predetermined amount of the mixed base substance was added to the ³⁵S-NF-κB decoy prepared above in small portions, to give a formulation A containing the NF-κB decoy in an amount of 0.4 mg (unlabelled NF-κB decoy content: 78.88%)/500 kBq/100 mg.

Aqueous formulation A (1 alginic acid formulation)

| ³⁵S-NF-kB decoy | 0.5% | 2.0 | mg |
|---|---|---|---|
| Alginic acid base substance | 99.5% | 398.0 | mg |
| (a suitable amount of 1% sodium | | | |
| alginate/sodium dihydrogen | | | |
| phosphate solution) | | | |
| Total | 100.0% | 400.0 | mg |

A certain amount of alginic acid base substance was added to and dissolved in the crude ³⁵S-NF-κB decoy; the mixture was agitated sufficiently in a Voltex mixer and then dispersed uniformly by pipetting with a micropipette, to give an aqueous formulation A containing ³⁵S-NF-κB decoy in an amount of 0.4 mg (unlabelled NF-κB decoy content: 78.88%)/500 kBq/100 mg.

The following media were used in preparing the dosing formulations: stearyl alcohol (Lot No. 20714C, listed in Japanese Pharmacopoeia, NOF Corporation), white vaseline (Lot No. 662275, listed in Japanese Pharmacopoeia, Nikko Pharmaceutical Co., Ltd.), purified hydrogenated soy bean phospholipid (Lot No. 3228, NIKKOL resinol S-10E, Nikko Chemicals Co., Ltd.), isopropyl myristate (Lot No. WAE5335, chemical use, Wako Pure Chemical Industries, Ltd.), distilled water for injection (Lot No. 3B76N, listed in Japanese Pharmacopoeia, Otsuka Pharmaceutical Co., Ltd.), and alginic acid base substance (a suitable amount of aqueous 1% sodium alginate/sodium dihydrogen phosphate solution).

(2) Test Animal

Seven-week-old male Crj:CD(SD) rats were used at one rat per one group. Rats having a body weight in the range of 220 to 330 g were used.

(3) Preparation of Coating Site

A rat under weak anesthesia with ether was shaved with an electric hair clippers or shaver carefully not to damage the skin the day before administration. The shaved site was examined on the day of administration, and it was confirmed that there was no inflammation or flare. The skin was designated as “normal skin”. A cellophane tape was pressed on and peeled from the shaved site ten times on the day of administration, and the skin where the cornified layer was peeled off was designated as “damaged skin”. The test animals were used, as divided as shown in Table 1.

TABLE 1

| Test Animal No. | Administered Formulation | Skin Condition |
|---|---|---|
| 1 | Formulation A | Normal |
| 2 | ″ | Damaged |
| 3 | Aqueous Formulation A | Normal |
| 4 | ″ | Damaged |

(4) Coating and Protection of Coated Site

The ³⁵S-NF-κB decoy formulation was coated on the back skin to a coating area of 3 cm in width and 3 cm in length. The dosage of the formulation was 0.4 mg/head, the amount of the formulation administered was 100 mg/head, the radioactivity concentration of the administered formulation was 500 kBq/head, and the number of dose was one time.

Then, a box-shaped paper frame (4 cm in width×4 cm in length×1 cm in height) was placed around the coated site and fixed with an expandable adhesive tape (Elastopore, Nichiban Co., Ltd.), to protect the coated site. The coating period was 24 hours.

(5) Removal of Administered Formulation

The coated site was wiped with a sheet of absorbent cotton (4 cm×5 cm) previously wetted with lukewarm water and squeezed tightly one time for a total of five times without force.

(6) Measurement of Skin Radioactivity Concentration

The animal after a single percutaneous administration of the ³⁵S-NF-κB decoy at a dosage of 0.4 mg/head was killed under ether anesthesia; the skin at the administered site was collected; and the radioactivity concentration of the skin was determined.

The radioactivity of the skin at the administered site was determined in the following manner: The skin at the administered site after removal of the dosing formulation was cut into a piece of the size of 2.5×2.5 cm from the center and of 2 mm in thickness, and frozen at around −20° C. as it is held slightly pressed between two slide glasses. Then, two sections having an area of 1 cm² were punched out from the dermis side of the frozen skin, and the dermis side after freezing was fixed onto a stage with an OCT compound under liquid nitrogen. After fixation, the skin having a thickness of 10 μm from the epidermal side was cut off with a cryostat while it is frozen. Four sections thus obtained (equivalent to 40 μm) were collected in a vial, heat-dissolved after addition of a tissue-solubilizing agent SOLUENE-350 (2 mL), and left together with 10 ml of a scintillator at room temperature; the radioactivity of the mixtures was determined in a liquid scintillation counter; and the radioactivity concentration of each layer was calculated from the values obtained. The formulation remaining at the coated site was wiped off 24 hours after application.

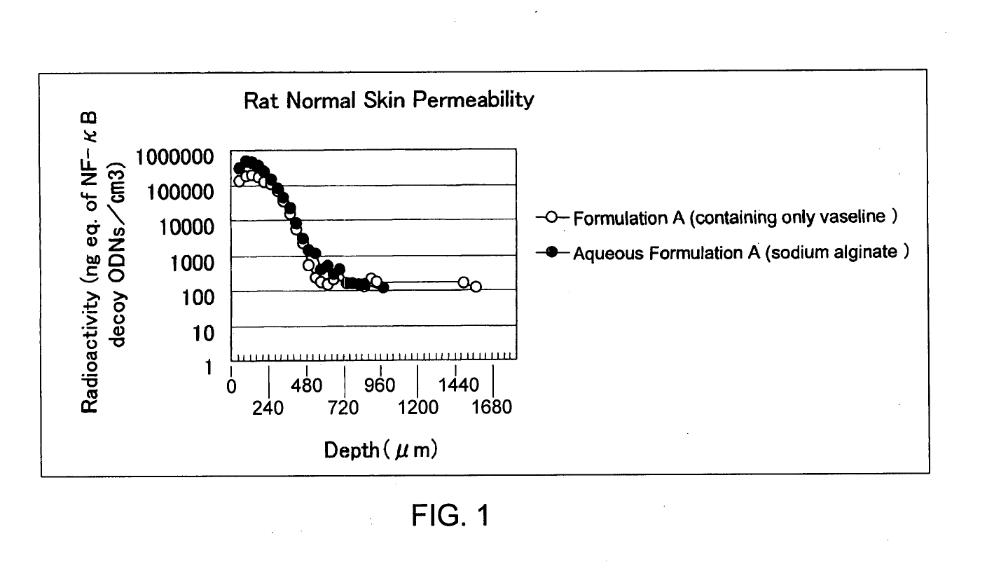

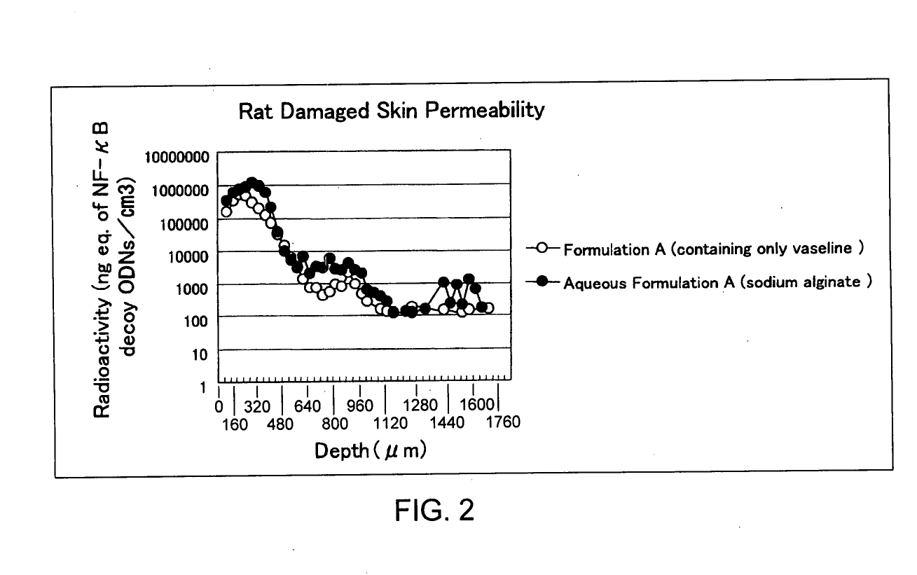

Results) Skin permeability test (see Tables 2 to 4 and FIGS. 1 and 2)

The results of the in-skin radioactivity concentration showed that a water-soluble gel, i.e., an aqueous formulation A (alginic acid formulation), was distinctively more effective in increasing skin permeability than the formulation A (single-vaseline formulation).

TABLE 2
Normal skin Radioactivity (ng eq. of NF-kB Decoy ODNs/cm³)

| Depth (μm) | Formulation A | Aqueous Formulation A |
|---|---|---|
| 40 | 127723 | 305858 |
| 80 | 176569 | 456038 |
| 120 | 195258 | 449339 |
| 160 | 160645 | 356230 |
| 200 | 122080 | 245652 |
| 240 | 103637 | 135583 |
| 280 | 67479 | 75834 |
| 320 | 34559 | 42958 |
| 360 | 14894 | 21062 |
| 400 | 5278 | 8044 |
| 440 | 2269 | 2884 |
| 480 | 533 | 1413 |
| 520 | 228 | 1130 |
| 560 | 165 | 379 |
| 600 | 150 | 492 |
| 640 | 202 | 285 |
| 680 | 0 | 399 |
| 720 | 0 | 163 |
| 760 | 0 | 163 |
| 800 | 0 | 143 |
| 840 | 125 | 143 |
| 880 | 220 | 0 |
| 920 | 165 | 0 |
| 960 | 0 | 118 |
| 1000 | 0 | 0 |
| 1040 | 0 | 0 |
| 1080 | 0 | 0 |
| 1120 | 0 | 0 |
| 1160 | 0 | 0 |
| 1200 | 0 | 0 |
| 1240 | 0 | 0 |
| 1280 | 0 | 0 |
| 1320 | 0 | 0 |
| 1360 | 0 | 0 |
| 1400 | 0 | 0 |
| 1440 | 0 | 0 |
| 1480 | 160 | 0 |
| 1520 | 0 | 0 |
| 1560 | 119 | 0 |
| 1600 | 0 | 0 |
| 1640 | 0 | 0 |
| 1680 | 0 | 0 |
| 1720 | 0 | 0 |
| 1760 | 0 | — |
| 1800 | 0 | — |

(Formulation A: only vaseline, Aqueous Formulation A:
sodium alginate, 0: detection limit or less, and —: data not
evaluated)

TABLE 3
Damaged skin Radioactivity(ng eq. of NF-kB Decoy ODNs/cm³)

| Depth ( pm) | Formulation A | Aqueous Formulation A |
|---|---|---|
| 40 | 151237 | 332272 |
| 80 | 328816 | 599862 |
| 120 | 496468 | 744654 |
| 160 | 482113 | 905723 |
| 200 | 300313 | 1227719 |
| 240 | 200318 | 958660 |
| 280 | 126664 | 602954 |
| 320 | 69206 | 214862 |
| 360 | 32528 | 38100 |
| 400 | 14366 | 10253 |
| 440 | 6132 | 5402 |
| 480 | 3148 | 3394 |
| 520 | 1360 | 6428 |
| 560 | 731 | 2011 |
| 600 | 753 | 3392 |
| 640 | 422 | 3124 |
| 680 | 546 | 5698 |
| 720 | 929 | 2787 |
| 760 | 779 | 2580 |
| 800 | 2136 | 4018 |
| 840 | 923 | 2534 |
| 880 | 465 | 2108 |
| 920 | 257 | 651 |
| 960 | 0 | 517 |
| 1000 | 150 | 381 |
| 1040 | 131 | 263 |
| 1080 | 0 | 121 |
| 1120 | 0 | 0 |
| 1160 | 0 | 132 |
| 1200 | 174 | 124 |
| 1240 | 0 | 0 |
| 1280 | 0 | 157 |
| 1320 | 0 | 0 |
| 1360 | 0 | 0 |
| 1400 | 144 | 991 |
| 1440 | 0 | 233 |
| 1480 | 0 | 879 |
| 1520 | 122 | 215 |
| 1560 | 144 | 1251 |
| 1600 | 0 | 609 |
| 1640 | 0 | 172 |
| 1680 | 148 | 0 |
| 1720 | 0 | 0 |
| 1760 | 0 | 0 |
| 1800 | 0 | 0 |

(Formulation A: only vaseline, Aqueous Formulation A: sodium alginate, 0: detection limit or less, —: and data not evaluated)

TABLE 4
Radioactivity concentration (% with respect to dosage)

| Formulation Type | Normal Skin | Damaged Skin |
|---|---|---|
| Formulation A | 9.18 (0-640 μm) | 20.36 (0-920 μm) |
| | 4.65 (121-640 μm) | 11.39 (121-800 μm) |
| Aqueous Formulation A | 19.30 (0-840 μm) | 52.62 (0-1080 μm) |
| | 8.19 (121-800 μm) | 37.03 (121-800 μm) |

(the value in the upper line of each formulation indicates the total radioactivity penetrated into the skin, while that in the lower line, the total radioactivity amount penetrated into the dermis).

A similar test on pig skin gave the following results.

TABLE 5
Radioactivity concentration (% with respect to dosage)

| Formulation Type | Normal Skin | Damaged Skin |
|---|---|---|
| Formulation A | 4.95 (0-880 μm) | 3.35 (0-640 μm) |
| | 2.83 (121-880 μm) | 2.73 (121-640 μm) |
| Aqueous Formulation A | 33.78 (0-1440 μm) | 41.59 (0-2440 μm) |
| | 22.57 (121-1440 μm) | 34.76 (121-2440 μm) |

(the value in the upper line of each formulation indicates the total radioactivity penetrated into the skin, while that in the lower line, the total radioactivity amount penetrated into the dermis).

The results above show that the aqueous formulation A (alginic acid formulation) is more preferable from the viewpoint of skin permeability.

It is possible to reduce the necessary amount of the NF-κB decoy sample to approximately 1/13 (0.15% concentration) of the conventional dosage, by using the aqueous formulation A.

2. Cumulative Skin Irritation Test

The cumulative skin irritation when only the aqueous formulation A containing ³⁵S-NF-κB decoy (alginic acid formulation) and an alginic acid base substance were administered onto the rabbit back skin for 14 days was studied.

(1) Test Animal

White female Japanese rabbits (Kbl: JW, SPF) shaved in the back were purchased from Minowa Plant of Kitayama Labes Co., Ltd. The rabbits were 17-week old and had body weight of 3.10 to 3.74 kg when the medicine was administered.

(2) Preparation of Coating Site and Coating

Two normal sites and damaged sites are formed on the back of each rabbit, and each substance was administered to at least six rabbits.

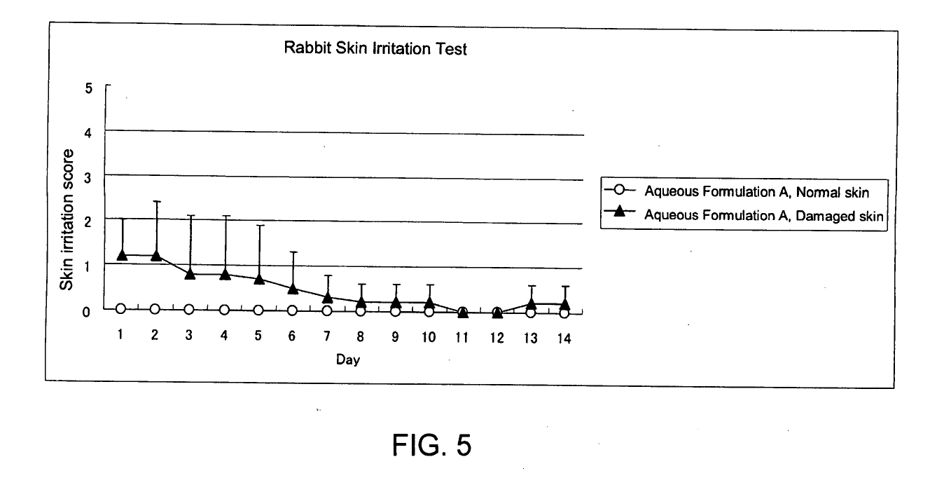

Fine hair in the back was shaved with an electric hair clipper the day before administration, and the four administration sites above were formed according to the Draize method (Draize, J. H. et al., J. Pharmacol. Exp. Ther., 82, 377-390 (1944)) (see FIG. 5). Two positions at point symmetry were used as normal skins, while the other two positions were shaved lightly with a shaver on the day of administration and used as damaged skins after stripping with a cellophane tape according to the Fukawa method (Kazunaga Fukawa et al., Journal of the Pharmaceutical Society of Japan 102, 89-98 (1982)). 0.2 g of each sample formulation A to D was weighed and spread uniformly over a lint cloth previously cut to a size of 2.5×2.5 cm; the cloth was applied on the skin; and a taping tape of 5×5 cm in size was mounted thereon to fix the lint cloth. The lint cloth was covered and fixed with a fabric cover (Stockinet, Alcare Co., Ltd.) and a tubular net bandage (Pressnet, Alcare Co., Ltd.) additionally. The aqueous formulation base substance and the aqueous formulation A were administered in an amount of 0.2 mL with a pipette and a spatula, while the penetration thereof into the skin is confirmed, and a lint cloth cut to a size of 2.5×2.5 cm and a taping tape of 5×5 cm in size were mounted thereon for fixation. It was also covered and fixed with a fabric cover and a tubular net bandage similarly. The lint cloth and the taping tape were removed after administration, and the administered site was washed with lukewarm water.

On the first day of administration, the alginic acid base material and the aqueous formulation A were administered at a dosage of 0.5 mL, but the amount was not sufficient for application of the area of 2.5×2.5 cm. Thus in the middle of the first day of administration, the dosage was changed respectively to the maximal administrable amounts of 0.2 g and 0.2 mL. On the first day of administration, 0.5 mL of the alginic acid base material was applied on the normal and damaged skins of animal number 1, and the aqueous formulation A on the normal skin in an amount of 0.5 mL and on the damaged skin in an amount of 0.2 mL. As for other animals, both the alginic acid base material and the aqueous formulation A were applied on the normal and damaged skins of other animals in an amount of 0.2 mL on the first day of administration.

(3) Judgment of Irritation

Skin irritation was observed before everyday administration and after the final administration (one hour after removal of administered substance) by visual observation. The judgment was made regarding erythema (incrustation) and edema at the administration site, according to the criteria of Draize. The judgment results were shown by the scores according to the Draize criteria. The averages and standard deviations of the erythema (incrustation) and edema and the total rate at each observation point respectively of the normal skin and the damaged skin were calculated. Low score means low irritation.

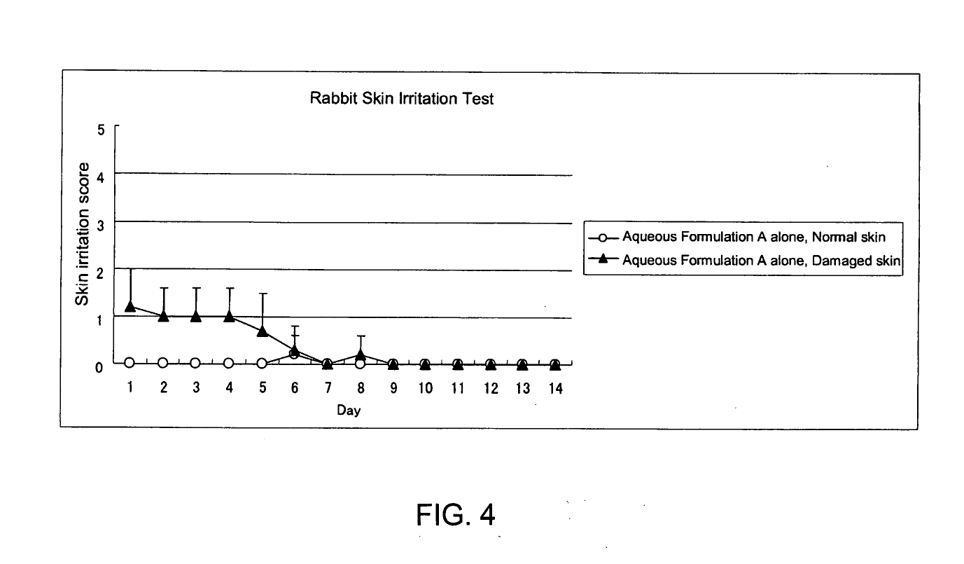

Results) The alginic acid base substance alone (aqueous formulation A alone) and the aqueous formulation A (alginic acid formulation) were not irritative to the normal skin of a rabbit. It caused erythema and edema on the damaged skin, but the degree was low, and continued administration only caused a temporary change that could be recovered. (see Table 6 and FIGS. 4 and 5.)

| | | Day | | | | | | | | | | | | | |
|---|---|---|---|---|---|---|---|---|---|---|---|---|---|---|---|
| Formulation | Skin | 1 | 2 | 3 | 4 | 5 | 6 | 7 | 8 | 9 | 10 | 11 | 12 | 13 | 14 |
| Aqueous Formulation A alone | Normal | 0.0 | 0.0 | 0.0 | 0.0 | 0.0 | 0.2 | 0.0 | 0.0 | 0.0 | 0.0 | 0.0 | 0.0 | 0.0 | 0.0 |
| SD | | 0.0 | 0.0 | 0.0 | 0.0 | 0.0 | 0.4 | 0.0 | 0.0 | 0.0 | 0.0 | 0.0 | 0.0 | 0.0 | 0.0 |
| Aqueous Formulation A alone | Damaged | 1.2 | 1.0 | 1.0 | 1.0 | 0.7 | 0.3 | 0.0 | 0.2 | 0.0 | 0.0 | 0.0 | 0.0 | 0.0 | 0.0 |
| SD | | 0.8 | 0.6 | 0.6 | 0.6 | 0.8 | 0.5 | 0.0 | 0.4 | 0.0 | 0.0 | 0.0 | 0.0 | 0.0 | 0.0 |
| Aqueous Formulation A | Normal | 0.0 | 0.0 | 0.0 | 0.0 | 0.0 | 0.0 | 0.0 | 0.0 | 0.0 | 0.0 | 0.0 | 0.0 | 0.0 | 0.0 |
| SD | | 0.0 | 0.0 | 0.0 | 0.0 | 0.0 | 0.0 | 0.0 | 0.0 | 0.0 | 0.0 | 0.0 | 0.0 | 0.0 | 0.0 |
| Aqueous Formulation A | Damaged | 1.2 | 1.2 | 0.8 | 0.8 | 0.7 | 0.5 | 0.3 | 0.2 | 0.2 | 0.2 | 0.0 | 0.0 | 0.2 | 0.2 |
| SD | | 0.8 | 1.2 | 1.3 | 1.3 | 1.2 | 0.8 | 0.5 | 0.4 | 0.4 | 0.4 | 0.0 | 0.0 | 0.4 | |

### INDUSTRIAL APPLICABILITY

Provided is a nucleic acid-based external skin formulation higher in skin permeability and lower in irritation.

What is claimed is:

1. A method for delivering a nucleic acid-based medicine to a patient having a skin disease comprising the step of administering dermally or epidermally a composition containing a nucleic acid-based medicine, sodium alginate and sodium dihydrogen phosphate.

2. The method of claim 1, wherein the nucleic acid-based medicine is a decoy molecule, antisense, ribozyme, aptamer or siRNA.

3. The method of claim 2, wherein the nucleic acid-based medium is a decoy.

4. The method of claim 3, wherein the decoy is NF-κB, STAT-1, GATA-3, STAT-6, AP-1, ETs or E2F decoy oligonucleotide.

5. The method of claim 4, wherein the skin disease is atopic dermatitis, contact dermatitis, photosensitive dermatitis, appendicular chronic dermatitis, seborrheic dermatitis, nummular dermatitis, systemic exfoliative dermatitis, stasis dermatitis, local abrasion dermatitis, medicamentosus dermatitis or psoriasis.

6. The method of claim 5, wherein the skin disease is atopic dermatitis, contact dermatitis, photosensitive dermatitis, appendicular chronic dermatitis, seborrheic dermatitis, nummular dermatitis, systemic exfoliative dermatitis, stasis dermatitis, local abrasion dermatitis, medicamentosus dermatitis or psoriasis.

7. The method of claim 6, wherein the skin disease is atopic dermatitis, contact dermatitis, photosensitive dermatitis, appendicular chronic dermatitis, seborrheic dermatitis, nummular dermatitis, systemic exfoliative dermatitis, stasis dermatitis, local abrasion dermatitis, medicamentosus dermatitis or psoriasis.

8. The method of claim 7, wherein the skin disease is atopic dermatitis, contact dermatitis, photosensitive dermatitis, appendicular chronic dermatitis, seborrheic dermatitis, nummular dermatitis, systemic exfoliative dermatitis, stasis dermatitis, local abrasion dermatitis, medicamentosus dermatitis or psoriasis.

9. The method of claim 8, wherein the skin disease is atopic dermatitis, contact dermatitis, photosensitive dermatitis, appendicular chronic dermatitis, seborrheic dermatitis, nummular dermatitis, systemic exfoliative dermatitis, stasis dermatitis, local abrasion dermatitis, medicamentosus dermatitis or psoriasis.
